(19) United States
(12) Patent Application Publication
(10) Pub. No.: US 2026/0199099 A1
(43) Pub. Date: Jul. 16, 2026

(54) METACARPAL FIXATION SCREW

(71) Applicant: DEV4 LLC, Houston, TX (US)

(72) Inventors: Lance TERRILL, Houston, TX (US); Jeff SEAVEY, Houston, TX (US)

(21) Appl. No.: 19/437,688

(22) Filed: Dec. 31, 2025

Related U.S. Application Data
(60) Provisional application No. 63/743,854, filed on Jan. 10, 2025.

(51) Int. Cl. A61F 2/42; A61B 17/72; A61F 2/30

(52) U.S. Cl. CPC A61F 2/4241; A61B 17/725; A61B 17/7291; A61F 2/30771; A61F 2002/30326; A61F 2002/30327; A61F 2002/30622; A61F 2002/30851; A61F 2002/30859

(57) ABSTRACT

A metacarpal fixation screw includes a cylindrical shaft and a thread. The cylindrical shaft includes a shaft tip at a leading end of the cylindrical shaft, a shaft head at an opposite trailing end of the cylindrical shaft, and an intermediate shaft body extending between and interconnecting the shaft tip and the shaft head. The thread extends along an outer surface of the cylindrical shaft from the leading end to the trailing end.

### PRIORITY CLAIM

This application claims priority under 35 U.S.C. § 119(e) to U.S. Provisional Ser. No. 63/743,854 , filed Jan. 10, 2025, which is expressly incorporated by reference herein in its entirety.

### BACKGROUND

The present disclosure relates to an orthopedic device, and particularly to a metacarpal device. More particularly, the present disclosure relates to a metacarpal fixation screw.

### SUMMARY

According to the present disclosure, a metacarpal fixation screw includes a cylindrical shaft and a thread. The cylindrical shaft includes a shaft tip at a leading end of the cylindrical shaft, a shaft head at an opposite trailing end of the cylindrical shaft, and an intermediate shaft body extending between and interconnecting the shaft tip and the shaft head. The thread extends along an outer surface of the cylindrical shaft from the leading end to the trailing end.

In illustrative embodiments, the metacarpal fixation screw further includes a cutting unit configured to assist with driving the metacarpal fixation screw into the medullary cavity of a metacarpal and removal of the metacarpal fixation screw from the medullary cavity of the metacarpal. The cutting unit includes a first plurality of cutting slots formed in the shaft tip and a second plurality of cutting slots formed in the thread. The first plurality of cutting slots are configured to cut pre-fixation intramedullary bone during insertion of the metacarpal fixation screw into the metacarpal. The second plurality of cutting slots are configured to reduce hoop stresses in the intramedullary bone during insertion of the metacarpal fixation screw and/or are configured to cut post-fixation intramedullary bone during removal of the metacarpal fixation screw from the metacarpal.

In illustrative embodiments, the thread includes a first portion or segment and a second portion or segment spaced apart from the first portion and connected to the first portion at a common lead point along the length of the metacarpal fixation screw. The first portion has a first pitch while the second portion has a second pitch less than the first pitch. In some embodiments, the second pitch is about half of the first pitch.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIG. 1 is a perspective view of a metacarpal fixation screw, in accordance with the present disclosure, including a cylindrical shaft and a thread coupled to an outer surface of the cylindrical shaft;

FIG. 2 is a cross section taken along line 2-2 in FIG. 1;

FIG. 3 is an enlarged view of a portion of FIG. 2;

FIG. 4 is a top plan view of the metacarpal fixation screw;

FIG. 5 is a bottom plan view of the metacarpal fixation screw; and

FIG. 6 is a perspective view of another embodiment of a metacarpal fixation screw including a double thread, in accordance with the present disclosure.

### DETAILED DESCRIPTION

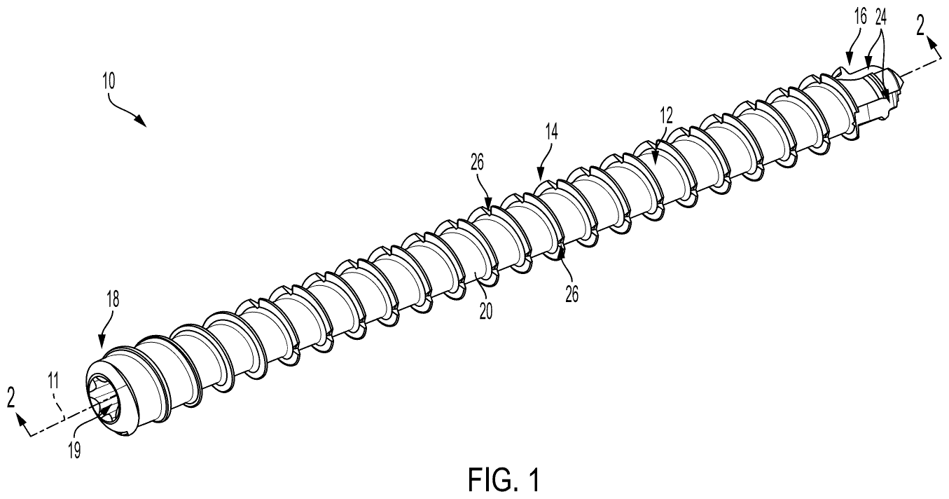

A metacarpal fixation screw 10, also called an intramedullary screw, is shown in FIG. 1. The metacarpal fixation screw 10 is configured to be inserted into a medullary cavity of a metacarpal to fixate and set a metacarpal fracture in the metacarpal. While the illustrative fixation screw 10 may be used to fixate a metacarpal fracture, it should be appreciated that the fixation screw can be used in any long bone or series of bones to fixate or set a fracture thereof.

The metacarpal fixation screw 10 includes a cylindrical shaft 12 and a thread 14 coupled to the cylindrical shaft 12. The cylindrical shaft 12 includes a shaft tip 16 at a leading end of the cylindrical shaft 12, a shaft head 18 at an opposite trailing end of the cylindrical shaft 12, and an intermediate shaft body 20 extending between and interconnecting the shaft tip 16 and the shaft head 18. The shaft head 18 is formed to include a drive cavity 19 configured to receive a tool or bit used to rotate the screw 10 about a longitudinal axis 11 for insertion or removal. The thread 14 extends along an outer surface of the cylindrical shaft 12 from the leading end to the trailing end to cause insertion or removal of the screw 10 in response to rotation about the longitudinal axis 11. In the illustrative embodiment, the cavity 19 extends all the way through the metacarpal screw 10 from the leading end to the trailing end but the metacarpal screw 10 may not be hollow in some embodiments.

In illustrative embodiments, the metacarpal fixation screw 10 further includes a cutting unit configured to assist with driving the metacarpal fixation screw 10 into the medullary cavity of a metacarpal and removal of the metacarpal fixation screw 10 from the medullary cavity of the metacarpal. The cutting unit includes a first plurality of cutting slots 24 formed in the shaft tip 16 and a second plurality of cutting slots 26 formed in the thread 14. The first plurality of cutting slots 24 are configured to cut pre-fixation intramedullary bone during insertion of the metacarpal fixation screw 10 into the metacarpal. The second plurality of cutting slots 26 are configured to cut post-fixation intramedullary bone during removal of the metacarpal fixation screw 10 from the metacarpal.

In the illustrative embodiment, the first plurality of cutting slots 24 includes a total of three cutting slots although more or less cutting slots may also be included. Each of the cutting slots 24 is defined by a cutting surface 25 and a lead surface 27 as shown in FIG. 5. The cutting surface 25 extends in a radial direction and is substantially perpendicular to the longitudinal axis of the metacarpal fixation screw 10. The cutting surface 25 faces in a clockwise direction 100. The cutting surface 25 is configured to trim bone from the metacarpal as the metacarpal fixation screw 10 is rotated in the clockwise direction about the longitudinal axis of the metacarpal fixation screw 10 during insertion into a metacarpal. The cutting surface 25 does not cut bone when the metacarpal fixation screw 10 is rotated in an opposite counter-clockwise direction 102. The lead surface 27 extends in the clockwise direction away from each corresponding cutting surface 25 and is spaced radially from the longitudinal axis of the metacarpal fixation screw 10. The lead surface 27 does not cut bone during insertion or removal of the metacarpal fixation screw 10 into or out of the metacarpal. The slots 24 extend through both the thread 14 and into the cylindrical shaft 12.

In the illustrative embodiment, the second plurality of cutting slots 26 includes a total of three cutting slots although more or less cutting slots may also be included. Each of the cutting slots 26 is defined by a cutting surface 28 and a lead surface 29. The cutting surface 28 extends in a radial direction and is substantially perpendicular to the longitudinal axis of the metacarpal fixation screw 10. The cutting surface 28 faces in the counter-clockwise direction and opposite to the cutting surfaces 25. The cutting surface 28 is configured to trim bone from the metacarpal as the metacarpal fixation screw 10 is rotated in the counter-clockwise direction about the longitudinal axis of the metacarpal fixation screw 10 during removal of the metacarpal fixation screw 10 from the metacarpal. The cutting surface 28 does not cut bone when the metacarpal fixation screw 10 is rotated in the clockwise direction. The lead surface 29 extends in the counter-clockwise direction away from each corresponding cutting surface 28 and is spaced radially from the longitudinal axis of the metacarpal fixation screw 10. The lead surface 29 does not cut bone during insertion or removal of the metacarpal fixation screw 10 into or out of the metacarpal. The cutting slots 26 provide a reverse cutting function to aid in removal of the screw 10 from the metacarpal and also reduce hoop stresses in the metacarpal fixation screw 10 by providing disruptions in the thread 14 where bone fragments can enter and pass longitudinally along the screw 10 during insertion and/or removal. The slots 26 extend only through the thread 14 and not into the cylindrical shaft 12.

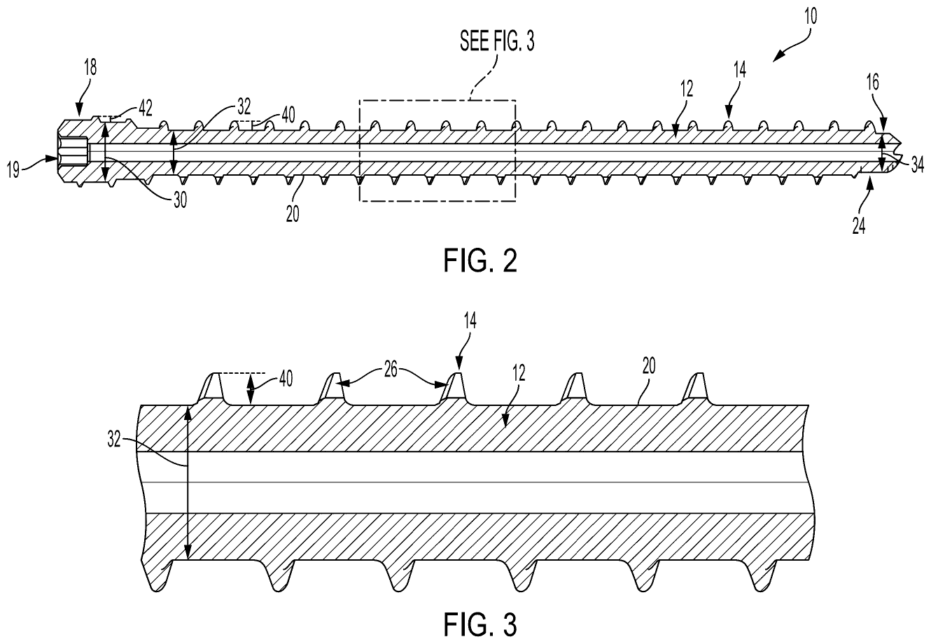

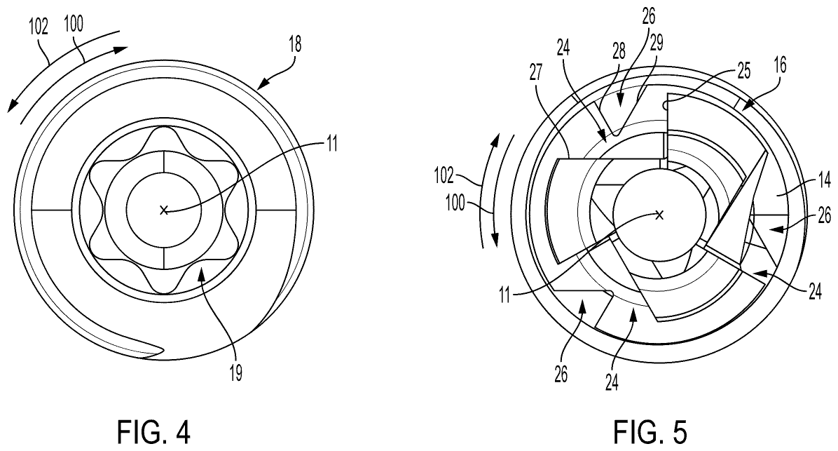

In the illustrative embodiment shown in FIGS. 1-5, the thread 14 is substantially continuous from the leading end to the trailing end and has a constant pitch from the leading end to the trailing end. In some embodiments, the pitch of the thread 14 is within a range of about 1.5 mm to about 3 mm. The thread 14 has a first depth 40 at the intermediate shaft body 20 and a second depth 42, less than the first depth 40, at the shaft head 18. In some embodiments, the depths 40, 42 are no more than 0.6 mm. In some embodiments, the thread 14 has a constant outer diameter along at least the shaft body 20 and in some cases along both the shaft body 20 and the head 18.

The intermediate shaft body 20 has a first diameter 30 and the shaft head 18 has a second diameter 32 greater than the first diameter 30. In some embodiments, the shaft tip 16 has a third diameter 34 less than the first and second diameters 30, 32 and tapering toward the first diameter 30 where the shaft tip 16 joins the intermediate shaft body 20. In the illustrative embodiment, the first diameter 30 is constant along the entire length of the intermediate shaft body 20. In some embodiments, the first diameter 30 is within a range of about 2 mm to about 5 mm. In some embodiments, the first diameter 30 is about 2.5 mm. In some embodiments, the first diameter 30 is about 3 mm. In some embodiments, the first diameter 30 is about 3.5 mm. In some embodiments, the first diameter 30 is about 4 mm. In some embodiments, the first diameter 30 is about 4.5 mm. In some embodiments, the first diameter 30 is about 5 mm.

Some intramedullary nails and/or screws include varying diameters (i.e. major and/or minor diameters) along the shaft length and/or varying thread depths/diameters. Such features can impart higher loads on the metacarpal during insertion and removal, and can cause further metacarpal fracture during a fracture fixation procedure. The metacarpal fixation screw 10 in the illustrative embodiment, has a substantially constant diameter and thread depth along substantially the entire length of the shaft body 20 except for the cutting slots 24, 26. As used herein, the term substantially is meant to account for minor tolerances and measurement inaccuracies within a range of +/−0.5%.

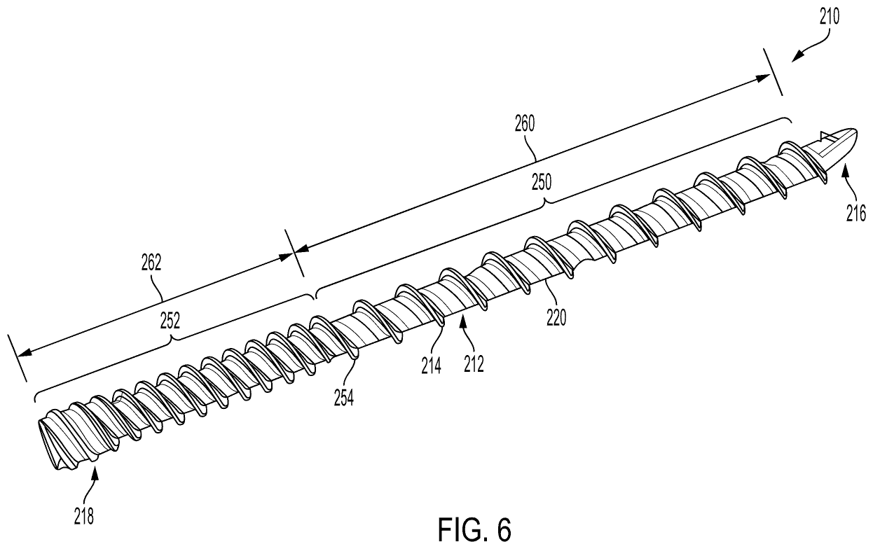

Another embodiment of a metacarpal fixation screw 210 is shown in FIG. 6. The metacarpal fixation screw 210 includes similar features to metacarpal fixation screw 10. For example, the metacarpal fixation screw 210 includes a cylindrical shaft 212 having a shaft tip 216, a shaft head 218, and an intermediate shaft body 220 extending between the shaft tip 216 and the shaft head 218. Accordingly, disclosure of metacarpal fixation screw 10 is hereby incorporated herein for metacarpal fixation screw 210 except for the differences between metacarpal fixation screws 10, 210 discussed below. It is further noted that features of both metacarpal fixations screws 10, 210 can be combined with one another.

The metacarpal fixation screw 210 further includes a thread 214 extending along a length of the cylindrical shaft 212. The thread 214 includes a first portion or segment 250 and a second portion or segment 252 spaced apart from the first portion 250 and connected to the first portion 250 at a point 254 along the length of the metacarpal fixation screw 210. The first portion 250 has a first pitch. The second portion 252 has a second pitch less than the first pitch. In some embodiments, the second pitch is half of the first pitch.

The first portion 250 extends from the shaft tip 216 to the point 254 on intermediate shaft body 220 between the shaft tip 216 and the shaft head 218. The second portion extends from the shaft head 218 to the point 254. The thread 214 has a common lead at the point 254.

The first portion 250 as a first length 260 and the second portion 252 has a second length 262. The second length 262 is about 30% to 50% of the first length 260. In some embodiments, the second length 262 is within a range of about 10 mm to about 20 mm. This distance corresponds with a typical or average metaphyseal length of the metacarpal. As used herein the term “about” means within 5% of the stated value, dimension, or percent.

1. An intramedullary fixation screw comprising:
a cylindrical shaft having a shaft tip at a leading end of the cylindrical shaft, a shaft head at an opposite trailing end of the cylindrical shaft, and an intermediate shaft body extending between and interconnecting the shaft tip and the shaft head, and
a thread extending along an outer surface of the cylindrical shaft from the leading end to the trailing end,
wherein the shaft tip is formed to include a first plurality of slots formed in the leading end and configured to cut pre-fixation intramedullary bone during insertion of the intramedullary fixation screw into an intramedullary space and the thread is formed to include a second plurality of slots configured to reduce hoop stresses in the intramedullary bone during insertion of the intramedullary fixation screw into the intramedullary space, and
wherein the thread is substantially continuous from the leading end to the trailing end and has a constant pitch from the leading end to the trailing end.

2. The intramedullary fixation screw of claim 1, wherein the second plurality of slots are configured to cut post-fixation intramedullary bone during removal of the fixation screw from the intramedullary space.

3. The intramedullary fixation screw of claim 1, wherein the intermediate shaft body has a first diameter and the shaft head has a second diameter greater than the first diameter.

4. The intramedullary fixation screw of claim 3, wherein the first diameter is constant along a length of the intermediate shaft body.

5. The intramedullary fixation screw of claim 3, wherein the thread has a first depth at the intermediate shaft body and a second depth, less than the first depth, at the shaft head.

6. The intramedullary fixation screw of claim 1, wherein the thread has a first pitch along a first portion of the intermediate shaft body and a second pitch less than the first pitch along a second portion of the intermediate shaft body.

7. The intramedullary fixation screw of claim 6, wherein the first portion extends from the shaft tip to a point on intermediate shaft body between the shaft tip and the shaft head, and the second portion extends from the shaft head to the point.

8. The intramedullary fixation screw of claim 7, wherein the thread has a common lead at the point.

9. The intramedullary fixation screw of claim 7, wherein the second pitch is about half of the first pitch.

10. The intramedullary fixation screw of claim 7, wherein a length of the second portion is within a range of about 10 mm to about 20 mm.

11. The intramedullary fixation screw of claim 7, wherein the first portion as a first length and the second portion has a second length, and the second length is about 30% to about 50% of the first length.

12. An intramedullary fixation screw comprising:
a cylindrical shaft having a shaft tip at a leading end of the cylindrical shaft, a shaft head at an opposite trailing end of the cylindrical shaft, and an intermediate shaft body extending between and interconnecting the shaft tip and the shaft head, and
a thread extending along an outer surface of the cylindrical shaft from the leading end to the trailing end,
wherein the thread has a first pitch along a first portion of the intermediate shaft body and a second pitch less than the first pitch along a second portion of the intermediate shaft body, and
wherein the thread has a common lead between the first portion and the second portion.

13. The intramedullary fixation screw of claim 12, wherein the intermediate shaft body has a first diameter and the shaft head has a second diameter greater than the first diameter.

14. The intramedullary fixation screw of claim 13, wherein the first diameter is constant along a length of the intermediate shaft body.

15. The intramedullary fixation screw of claim 13, wherein the thread has a first depth at the intermediate shaft body and a second depth, less than the first depth, at the shaft head.

16. The intramedullary fixation screw of claim 12, wherein the second pitch is half of the first pitch.

17. The intramedullary fixation screw of claim 12, wherein a length of the second portion is within a range of about 10 mm to about 20 mm.

18. The intramedullary fixation screw of claim 12, wherein the first portion as a first length and the second portion has a second length, and the second length is about 30% to about 50% of the first length.

19. The intramedullary fixation screw of claim 12, further comprising a cutting unit including a first plurality of cutting slots formed in the leading end and configured to cut pre-fixation intramedullary bone during insertion of the metacarpal fixation screw into an intramedullary space and a second plurality of cutting slots formed in the thread and configured to cut post-fixation intramedullary bone during removal of the metacarpal fixation screw from the intramedullary space.

20. The intramedullary fixation screw of claim 12, wherein the intermediate shaft body has a constant minor diameter and the thread has a constant major diameter along a length of the intermediate shaft body.
